(19) United States
(12) Patent Application Publication
(10) Pub. No.: US 2026/0335329 A1
(43) Pub. Date: Oct. 29, 2026

(54) PROTEIN PHOSPHATASE AGONIST AND USE

(71) Applicant: NANJING CHINRILY MEDICA CO. LTD., Nanjing, Jiangsu (CN)

(72) Inventors: Guoqiang SU, Nanjing (CN); Jiabin YANG, Nanjing (CN); Ya ZHANG, Nanjing (CN); Haowei XU, Nanjing (CN); Bolang SU, Nanjing (CN); Yuxing SU, Nanjing (CN)

(73) Assignee: NANJING CHINRILY MEDICA CO. LTD., Nanjing, Jiangsu (CN)

(21) Appl. No.: 19/110,347

(22) PCT Filed: Sep. 9, 2022
(86) PCT No.: PCT/CN2022/117987

§ 371 Date: Oct. 1, 2025

(51) Int. Cl. A61K 31/5365; C07D 498/04

(52) U.S. Cl. CPC A61K 31/5365; C07D 498/04

(57) ABSTRACT

A benzopyrazolooxazine derivative of a structure shown in formula I, or a pharmaceutically acceptable salt thereof. R is selected from hydrogen, halogen, alkyl, or alkoxy; m is an integer from 1 to 3; X is selected from oxygen or sulfur; and R₁ is selected from hydrogen, alkyl, or acyl. Further, use of the benzopyrazolooxazine derivative in preparation of a medicament for prevention and treatment of a disease caused by lack of PP2A activity or reduction of PP2A functions. Also, use of the benzopyrazolooxazine derivative in preparation of a protein phosphatase agonist.

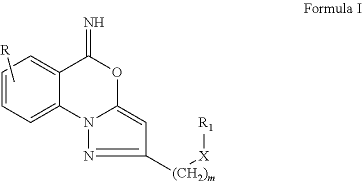

### TECHNICAL FIELD

The present invention belongs to the field of medicinal chemistry, and relates to a protein phosphatase agonist and use, and specifically, to a benzopyrazolooxazine protein phosphatase agonist and use.

### BACKGROUND

Protein phosphatase 2A (PP2A) is an important member of the protein phosphatase family. The catalytic domain of PP2A was highly homologous with the other members of this family. PP2A mediated the whole process of cellular life activities, such as cell signal transduction, cell differentiation and apoptosis.

The important downstream pathways of PP2A, including MAPK and ERK, were blocked by the loss or reduction of PP2A activity, which induced cell regulation disorder and carcinogenesis. In T-cells, the loss of PP2A activity could decrease their clearance ability. That might play an important role in the cellular carcinogenesis.

The release of neurotransmitters and the opening and closing of ion channels, were playing an important role in the process of information transmission within nerve cells, which was regulated by the protein phosphorylation and dephosphorylation. Under the action of adenosine triphosphate (ATP) and protein phosphorylase (protein kinase, PK), protein phosphorylation was catalyzed, the protein structure changed and the proteins transformed to their activated form. The activated protein continued to activate the downstream target proteins, further activated the whole cascade reaction and completed signal transduction. PP2A was the key factor for protein dephosphorylation, ensuring the signal pathway could be closed in time for the next neural activity.

Alzheimer's disease (AD), also known as Senile Dementia, was a neurological disorder characterized by memory, cognitive, language and behavioral impairments. It was caused by chronic and progressive degenerative changes in the central nervous system.

According to the statistics of China Alzheimer's disease report 2021 headed by Shanghai Ruijin Hospital, the number of existing patients with AD and other dementias in China reached 13243950 in 2019. The prevalence rate and mortality rate were slightly higher than the global average, and the relevant data in women were higher than that in men. The prevalence rate (1188.9/100000) and mortality rate (30.8/100000) of women were higher than that of men (669.3/100000) and mortality rate (14.6/100000), respectively.

Currently, two types of drugs were widely used in the clinical treatment of AD, cholinesterase inhibitors such as donepezil and N-methyl-D-aspartate (NMDA) receptor antagonists such as memantine. Donepezil could increase cholinergic nerve signals by inhibiting acetylcholinesterase. The patients' cognitive function were improved through maintaining or increasing acetylcholine concentration at a certain level. On the other hand, memantine reduced the neurotoxic effect of excitatory amino acids and improve cognitive function by inhibiting NMDA receptor activity. However, these drugs were only symptomatic treatments, which could improve cognitive and memory impairment. So far, there were no drugs that could prevent or delay the development of AD.

The growth and progress of AD were closely related to the decreased function of PP2A. When the activity of PP2A was reduced, the protein losed activity because of the failure of dephosphorylate, the postsynaptic membrane continued to receive neurotransmitters that released by the presynaptic membrane. The signal channels couldn't close in time which resulted in nerve conduction delay and memory failure.

Amyloid precursor protein (APP) is an essential bioactive substance that widely exists in the body. β-amyloid (Aβ) is produced by hydrolysis of app by β-secretase and γ-secretase. In vitro test results showed that Aβ possed inhibitory effect on the biological activity of protein phosphatase (PP), and its activity inhibition intensity was closely in correlation with the level of Aβ. The normal level of Aβ showed limited effect on PP activity. When the Aβ reached a suitable concentration, the activity of PP would be inhibited. The inhibition of PP activity could be enhanced by increasing Aβ concentration. In conclusion, the sharply increasing of Aβ level in the body inhibited PP activity, which played an extremely important role in the growth of AD.

The biological function of tau protein (p-tau) was to induce tubulins polymerization into microtubules and prevent their depolymerization, thereby maintaining the morphology and function of nerve cells. In nerve cells, in order to revive PP activity, p-tau tangled with Aβ to form p-tau/Aβ complexes, then the complexes deposited in the cells and reduced Aβ concentration, which could reduce the level of Aβ and revive the activity of PP. The fluctuating memory of AD patients is caused by the fluctuation of PP biological activity with the level of Aβ. Eventually, the P-tau/Aβ complex broke down nerve cells and caused irreversible organic lesions in the nerves of AD patients. PET scans of AD patients showed that P-tau/Aβ tangles (plaques) caused brain atrophy and death of brain nerve cells.

PP Agonist could improve the activity of PP and catalyze the dephosphorylation of protein to maintain normal neural activity. In addition, increased PP activity could also promote the dephosphorylation of phosphorylated tau protein (P-tau-Pi), thereby avoiding the neurotoxic effects and nerve cells apoptosis mediated by excessive phosphorylation of tau protein.

Therefore, development of novel PP2A agonists will bring hope to the patients with diseases, such as cancer and AD, which caused by reduced PP2A activity.

### SUMMARY

An objective of the present invention is to provide a benzopyrazolooxazine compound. The compound can be used as a protein phosphatase agonist with a strong excitatory effect on PP2A with impaired activity, and can significantly increase PP2A bioactivity.

Provided is a benzopyrazolooxazine derivative of a structure shown in formula I, or a pharmaceutically acceptable salt thereof:

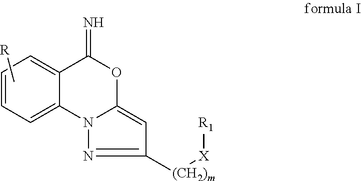

where
R is selected from hydrogen, halogen, alkyl, or alkoxy;
m is an integer from 1 to 3;
X is selected from oxygen or sulfur; and
R₁ is selected from hydrogen, alkyl, or acyl.
Preferably, R is selected from hydrogen, halogen, C₁-C₂ alkyl, or C₁-C₂ alkoxy;
m is an integer from 1 to 3;
X is selected from oxygen or sulfur; and
R₁ is selected from hydrogen, C₁-C₃ alkyl, formyl (—CHO), or acetyl (—COCH₃).

The halogen is specifically selected from F or Cl. The alkyl may be specifically selected from methyl, ethyl, n-propyl, or the like. The alkoxy may be specifically selected from methoxy, ethoxy, or the like.

The benzopyrazolooxazine derivative is preferably selected from, but is not limited to, the following compounds:
(5-imino-5H-benzo[d]pyrazolo[5,1-b][1,3]oxazin-2-yl) methanol;
2-(methoxymethyl)-5H-benzo[d]pyrazolo[5,1-b][1,3]oxazin-5-imine;
2-(ethoxymethyl)-5H-benzo[d]pyrazolo[5,1-b][1,3]oxazin-5-imine;
2-(propoxymethyl)-5H-benzo[d]pyrazolo[5,1-b][1,3]oxazin-5-imine;
(5-imino-5H-benzo[d]pyrazolo[5,1-b][1,3]oxazin-2-yl)methyl formate;
(5-imino-5H-benzo[d]pyrazolo[5,1-b][1,3]oxazin-2-yl)methyl acetate;
2-((methylthio)methyl)-5H-benzo[d]pyrazolo[5,1-b][1,3]oxazin-5-imine;
2-(methoxymethyl)-8-methyl-5H-benzo[d]pyrazolo[5,1-b][1,3]oxazin-5-imine;
2-(methoxymethyl)-8-ethyl-5H-benzo[d]pyrazolo[5,1-b][1,3]oxazin-5-imine;
2-(methoxymethyl)-8-fluoro-5H-benzo[d]pyrazolo[5,1-b][1,3]oxazin-5-imine;
2-(methoxymethyl)-7-fluoro-5H-benzo[d]pyrazolo[5,1-b][1,3]oxazin-5-imine;
2-(methoxymethyl)-7-chloro-5H-benzo[d]pyrazolo[5,1-b][1,3]oxazin-5-imine;
2-(methoxymethyl)-7-methoxy-5H-benzo[d]pyrazolo[5,1-b][1,3]oxazin-5-imine;
2-(methoxymethyl)-7-ethoxy-5H-benzo[d]pyrazolo[5,1-b][1,3]oxazin-5-imine;
2-(ethoxymethyl)-7-fluoro-5H-benzo[d]pyrazolo[5,1-b][1,3]oxazin-5-imine;
2-(propoxymethyl)-7-fluoro-5H-benzo[d]pyrazolo[5,1-b][1,3]oxazin-5-imine;
2-(methoxyethyl)-8-fluoro-5H-benzo[d]pyrazolo[5,1-b][1,3]oxazin-5-imine; and
2-(methoxypropyl)-8-fluoro-5H-benzo[d]pyrazolo[5,1-b][1,3]oxazin-5-imine.

The pharmaceutically acceptable salt is a salt, in a general sense, that has no side effects and can improve physicochemical properties (for example, water solubility) of the compound, which is formed through a reaction between the benzopyrazolooxazine derivative and a pharmaceutically common acid or base. The acid is selected from hydrochloric acid, hydrobromic acid, sulfuric acid, nitric acid, phosphoric acid, succinic acid, maleic acid, fumaric acid, acetic acid, citric acid, tartaric acid, benzoic acid, benzenesulfonic acid, or naphthalenesulfonic acid. The base is selected from sodium hydroxide, sodium carbonate, sodium bicarbonate, potassium hydroxide, or the like.

An objective of the present invention is to provide a method for preparing a benzopyrazolooxazine derivative of a structure shown in formula I. When R₁ is selected from hydrogen or alkyl, a reaction route is as follows:

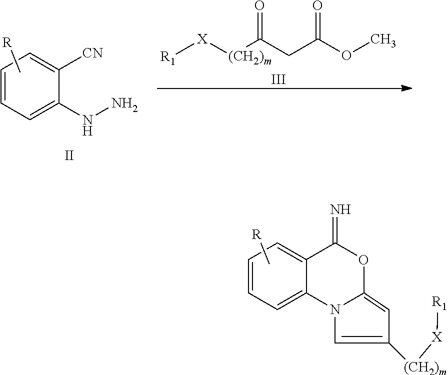

When R₁ is selected from acyl, a reaction route is as follows:

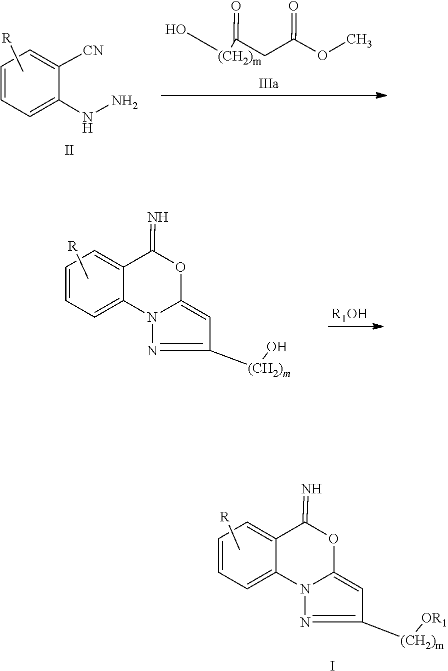

The compound II may be prepared by subjecting the compound IV to a diazotization reaction and a reduction reaction sequentially, with a reaction route as follows:

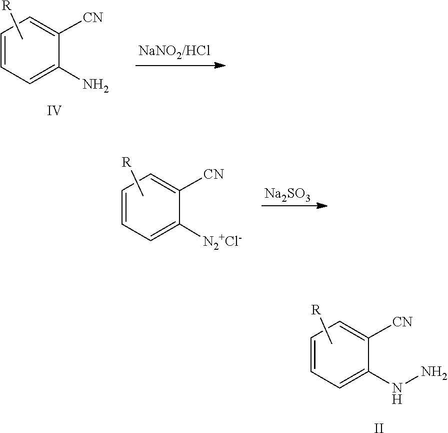

The compound II may alternatively be prepared through a reaction between the compound V and different concentrations of hydrazine solutions, hydrazine salts, or hydrazine derivatives with different substituents, with a reaction route as follows:

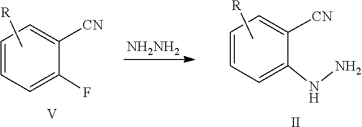

Y includes, but is not limited to, F, Cl, Br, I, and a sulfonic acid group.

Unless otherwise specified, the compound used as a raw material for preparing the benzopyrazolooxazine derivative shown in formula I in the present invention is a known compound that is commercially available, or may be prepared from a known compound by a known method, a method similar to a known method, or a method similar to the method in an embodiment.

The benzopyrazolooxazine derivative shown in formula I has a strong excitatory effect on PP2A with impaired activity, can significantly increase PP2A bioactivity, can significantly inhibit okadaic acid-induced phosphorylation of tau protein in cells, and can significantly increase a survival rate of cells with okadaic acid- or Aβ₁₋₄₂-induced injury.

Another objective of the present invention is to provide use of the benzopyrazolooxazine derivative shown in formula I in preparation of a medicament for prevention and treatment of a disease caused by lack of PP2A activity or reduction of PP2A functions.

Preferably, the disease caused by lack of PP2A activity or reduction of PP2A functions is a tumor. The benzopyrazolooxazine derivative shown in formula I can be used in prevention and treatment of the tumor to avoid formation and progression of the tumor.

Preferably, the disease caused by lack of PP2A activity or reduction of PP2A functions is a degenerative brain disease, and the degenerative brain disease includes, but is not limited to, Alzheimer's disease (AD). The benzopyrazolooxazine derivative shown in formula I can be used in prevention and treatment of the Alzheimer's disease.

Another objective of the present invention is to provide use of the benzopyrazolooxazine derivative shown in formula I in preparation of a protein phosphatase agonist, preferably in preparation of a protein phosphatase 2A agonist.

### DETAILED DESCRIPTION

### Experimental Instruments

Rotary evaporator RE-52AA (Shanghai Yarong Biochemical Instrument Factory); SHB-III water-circulation multi-purpose vacuum pump (Nanjing Wener Instrument Equipment Co., Ltd); JJ-1 precision power-increasing stirrer (Jintan Chengdong Xinrui Instrument Factory); 85-1 magnetic stirrer (Nanjing Wener Instrument Equipment Co., Ltd); ZF-I triple-purpose ultraviolet analyzer; X-4 digital melting point meter (Beijing Fukai Instrument Co., Ltd); Bruker AV-300 NMR spectrometer (with TMS as an internal standard and DMSO-de as a solvent); Agilent Technologies 6520B Accurate-Mass A-TOF LC/MS mass spectrometer; Shimadzu LC-20AT high-performance liquid chromatograph for measurement of a purity of a compound, Inertsustain C₁₈ chromatographic column, SAP-10 ultraviolet detector, detection wavelength 220 nm, mobile phases: methanol-water (70:30 V/V).

### Materials

Silica gel plate for thin-layer chromatography (silica gel GF254, Yantai Dexin Biology Science and Technology Co., Ltd). DMF was dried by using 4 Å molecular sieves, ethanol was dried by using 3 Å molecular sieves, and other reagents were commercially available and analytically or chemically pure. Most of the reagents were obtained from Aladdin Reagent Co., Ltd.

### Example 1

### Preparation of (5-imino-5H-benzo[d]pyrazolo[5,1-b][1,3]oxazin-2-yl) methanol (Compound 1)

### 1. Preparation of 2-cyanophenylhydrazine

2-cyanophenylhydrazine was prepared with reference to the method in Example 1 “preparation of 2-hydrazino-benzonitrile hydrochloride” in the Chinese Patent No. CN101508696A.

### 2. Preparation of (5-imino-5H-benzo[d]pyrazolo[5,1-b][1,3]oxazin-2-yl)methanol

To a reaction flask were added 1.3 g of 2-cyanophenylhydrazine crude product and 15 mL of saturated solution of hydrogen chloride in ethanol. 2.0 g of methyl 4-hydroxy-3-oxobutanoate was added under stirring, to react at room temperature overnight. 30 mL of ice water was added. The mixture was stirred for 1 h and then filtered. The filter cake was washed with water for multiple times, and then dried to give 1.7 g of crude compound 1 hydrochloride. The crude compound 1 hydrochloride was recrystallized with 10% ethanol to give 1.3 g of refined compound 1 hydrochloride (white solid).

To a reaction flask were added the refined compound 1 hydrochloride and 20 mL of water. The temperature was controlled below 15° C. A sodium hydroxide solution was added dropwise to adjust pH to 7-8. Then, the mixture was stirred for 1 h and then filtered. The filter cake was washed with water for multiple times, and then dried to give 0.6 g of compound 1.

ESI-MS: m/z 216.2 [M+1]⁺.

### Example 2

### Preparation of 2-(methoxymethyl)-5H-benzo[d]pyrazolo[5,1-b][1,3]oxazin-5-imine (Compound 2)

Compound 2 was obtained according to the method in Example 1 with methyl 4-methoxy-3-oxobutanoate that replaced methyl 4-hydroxy-3-oxobutanoate.

ESI-MS: m/z 230.2 [M+1]⁺.

### Example 3

### Preparation of 2-(ethoxymethyl)-5H-benzo[d]pyrazolo[5,1-b][1,3]oxazin-5-imine (Compound 3)

Compound 3 was obtained according to the method in Example 1 with methyl 4-ethoxy-3-oxobutanoate that replaced methyl 4-hydroxy-3-oxobutanoate.

ESI-MS: m/z 243.3 [M+1]⁺.

### Example 4

### Preparation of 2-(propoxymethyl)-5H-benzo[d]pyrazolo[5,1-b][1,3]oxazin-5-imine (Compound 4)

Compound 4 was obtained according to the method in Example 1 with methyl 4-propoxy-3-oxobutanoate that replaced methyl 4-hydroxy-3-oxobutanoate.

ESI-MS: m/z 258.3 [M+1]⁺.

### Example 5

### Preparation of (5-imino-5H-benzo[d]pyrazolo[5,1-b][1,3]oxazin-2-yl)methyl formate (Compound 5)

10 mL of formic acid was added to a reaction flask. The reaction flask was cooled down in an ice bath to −10° C. 2 mL of acetic anhydride was added dropwise. The mixture was stirred at the same temperature for 0.5 h. 0.5 g of compound 1 and 0.1 g of DMAP were added. The mixture was warmed to room temperature to react for 2 h. 50 mL of ice water was added. The mixture was filtered. The filter cake was washed with water until the washing buffer was neutral, and then recrystallized with ethyl acetate to give compound 5.

ESI-MS: m/z 244.2 [M+1]⁺.

### Example 6

### Preparation of (5-imino-5H-benzo[d]pyrazolo[5,1-b][1,3]oxazin-2-yl)methyl acetate (Compound 6)

Compound 6 was obtained according to the method in Example 5 with acetic acid that replaced formic acid.

ESI-MS: m/z 258.3 [M+1]⁺.

### Example 7

### Preparation of 2-((methylthio)methyl)-5H-benzo[d]pyrazolo[5,1-b][1,3]oxazin-5-imine (Compound 7)

### 1. Preparation of methyl 4-(methylthio)-3-oxobutanoate

To a reaction flask were added 500 mL of DMF, 108.5 g of methyl chloroacetate, and 72.1 g of sodium thiomethoxide sequentially to react at 105° C. for 5 h, and then the heating was stopped. The mixture was cooled down to room temperature. 2.5 L of water was added to the reaction mixture. An aqueous phase was extracted with dichloromethane (3×500 mL). Organic phases were combined and then washed with water (5×300 mL) and saturated brine (300 mL) sequentially. The combined organic phase was dried over anhydrous sodium sulfate and then concentrated under reduced pressure to give 89.2 g of oily substance, which was methyl 2-(methylthio)acetate. The methyl 2-(methylthio)acetate was not purified and was directly subjected to a next reaction.

The methyl 2-(methylthio)acetate and 118.9 g of diethyl malonate were dissolved in 300 mL of dried DMF. The mixture was added to a pressure-equalizing dropping funnel for use.

300 mL of dried DMF was added to a dry three-necked flask. 44.5 g of 60% sodium hydride was added in batches to the flask being cooled down in an ice-salt bath. The temperature was controlled at 0±5° C. The foregoing mixture was added dropwise and then kept at the same temperature to react for 1 h. Then, crushed ice was added to terminate the reaction. The reaction mixture was poured into a separatory funnel. 3 L of water was added. The mixture was subjected to extraction with dichloromethane (3×500 mL). Organic phases were combined and then washed with water (5×300 mL) and saturated brine (300 mL) sequentially. The combined organic phase was dried over anhydrous sodium sulfate and then concentrated under reduced pressure to give 143.3 g of oily substance, which was diethyl 2-(2-(methylthio) acetyl) malonate.

The diethyl 2-(2-(methylthio) acetyl) malonate was added to 1 L of ethanol. 50.8 g of sodium hydroxide was added under stirring. The resulting mixture was stirred at room temperature overnight and then filtered. The filter cake was washed with an appropriate amount of ethanol to give disodium 2-(2-(methylthio) acetyl) malonate. Then, the disodium 2-(2-(methylthio) acetyl) malonate was added to 1.5 L of ethanol. 150 mL of concentrated hydrochloric acid was added. The mixture was stirred for 1 h and then filtered. The filter cake was washed with an appropriate amount of ethanol. The filtrate was concentrated to give 67.6 g of 2-(2-(methylthio) acetyl) malonic acid.

To a reaction flask were added the 2-(2-(methylthio) acetyl) malonic acid and 1 L of xylene. After reflux for 2 h, the xylene was distilled out. 1 L of saturated solution of hydrogen chloride in ethanol was added to the residue. The mixture was stirred at room temperature overnight and then distilled under reduced pressure to remove the solvent. The residue was dissolved in 500 mL of ethyl acetate. The mixture was washed with a saturated sodium bicarbonate solution and then concentrated under reduced pressure to give 34.6 g of methyl 4-(methylthio)-3-oxobutanoate. The methyl 4-(methylthio)-3-oxobutanoate was not purified and was directly subjected to a next reaction.

### 2. Preparation of Compound 7

Compound 7 was obtained according to the method in Example 1 with methyl 4-methylthio-3-oxobutanoate that replaced methyl 4-hydroxy-3-oxobutanoate.

ESI-MS: m/z 246.3 [M+1]⁺.

### Example 8

### Preparation of 2-(methoxymethyl)-8-methyl-5H-benzo[d]pyrazolo[5,1-b][1,3]oxazin-5-imine (Compound 8)

### 1. Preparation of 5-methyl-2-cyanophenylhydrazine

To a reaction flask were added 67.6 g of 2-fluoro-4-methylbenzonitrile, 500 mL of tetrahydrofuran, and 500 mL of hydrazine hydrate. The mixture was stirred at room temperature for 24 h. An aqueous phase was removed. An organic phase was concentrated under reduced pressure until dried to give a 2-cyano-5-methylphenylhydrazine crude product. The 2-cyano-5-methylphenylhydrazine crude product was not purified and was directly subjected to a next reaction.

### 2. Preparation of Compound 8

Compound 8 was obtained according to the method in Example 1 with 2-cyano-5-methylphenylhydrazine that replaced 2-cyanophenylhydrazine and with methyl 4-methoxy-3-oxobutanoate that replaced methyl 4-hydroxy-3-oxobutanoate.

ESI-MS: m/z 244.3 [M+1]⁺.

### Example 9

### Preparation of 2-(methoxymethyl)-8-ethyl-5H-benzo[d]pyrazolo[5,1-b][1,3]oxazin-5-imine (Compound 9)

### 1. Preparation of 5-ethyl-2-cyanophenylhydrazine

To a reaction flask with a rubber stopper and a gas introduction tube were added 31.1 g of 2-fluoro-4-chlorobenzonitrile, 3.5 g of ferric acetylacetonate, and 300 mL of THF. The reaction flask was purged with N₂ for three times. Then, 150 mL of 2 mol/L EtMgBr-THF solution was injected by using a syringe through the rubber stopper. The mixture was stirred at room temperature for 0.5 h. Then, the reaction mixture was poured into 1.5 L of dilute hydrochloric acid. The resulting mixture was subjected to extraction with 500 mL of ethyl acetate. An organic phase was washed with 3×100 mL of water and 100 mL of saturated brine sequentially, dried over anhydrous sodium sulfate, and then distilled under reduced pressure to remove the solvent. The residue was subjected to flash chromatography to give 17.3 g of 2-fluoro-4-ethylbenzonitrile.

To a reaction flask were added 17.3 g of 2-fluoro-4-ethylbenzonitrile, 300 mL of tetrahydrofuran, and 300 mL of hydrazine hydrate. The mixture was stirred at room temperature for 24 h. An aqueous phase was removed. An organic phase was concentrated under reduced pressure until dried to give a 2-cyano-5-ethylphenylhydrazine crude product. The 2-cyano-5-ethylphenylhydrazine crude product was not purified and was directly subjected to a next reaction.

### 2. Preparation of Compound 9

Compound 9 was obtained according to the method in Example 1 with 5-ethyl-2-cyanophenylhydrazine that replaced 2-cyanophenylhydrazine and with methyl 4-methoxy-3-oxobutanoate that replaced methyl 4-hydroxy-3-oxobutanoate.

ESI-MS: m/z 258.3 [M+1]⁺.

### Example 10

### Preparation of 2-(methoxymethyl)-8-fluoro-5H-benzo[d]pyrazolo[5,1-b][1,3]oxazin-5-imine (Compound 10) 1. Preparation of 2-cyano-5-fluorophenylhydrazine

The 2-cyano-5-fluorophenylhydrazine was prepared with reference to the method in Example 1 in the Chinese Patent No. CN101508696A with 2-amino-4-fluorobenzonitrile that replaced 2-aminobenzonitrile.

### 2. Preparation of Compound 10

Compound 10 was obtained according to the method in Example 1 with 2-cyano-5-fluorophenylhydrazine that replaced 2-cyanophenylhydrazine and with methyl 4-methoxy-3-oxobutanoate that replaced methyl 4-hydroxy-3-oxobutanoate.

ESI-MS: m/z 248.2 [M+1]⁺.

### Example 11

### Preparation of 2-(methoxymethyl)-7-fluoro-5H-benzo[d]pyrazolo[5,1-b][1,3]oxazin-5-imine (Compound 11)

### 1. Preparation of 2-cyano-4-fluorophenylhydrazine

The 2-cyano-4-fluorophenylhydrazine was prepared with reference to the method in Example 1 in the Chinese Patent No. CN101508696A with 2-amino-5-fluorobenzonitrile that replaced 2-aminobenzonitrile.

### 2. Preparation of Compound 11

Compound 11 was obtained according to the method in Example 1 with 2-cyano-4-fluorophenylhydrazine that replaced 2-cyanophenylhydrazine and with methyl 4-methoxy-3-oxobutanoate that replaced methyl 4-hydroxy-3-oxobutanoate.

ESI-MS: m/z 248.2 [M+1]⁺.

### Example 12

### Preparation of 2-(methoxymethyl)-7-chloro-5H-benzo[d]pyrazolo[5,1-b][1,3]oxazin-5-imine (Compound 12)

### 1. Preparation of 2-cyano-4-chlorophenylhydrazine

The 2-cyano-4-chlorophenylhydrazine was prepared with reference to the method in Example 1 in the Chinese Patent No. CN101508696A with 2-amino-5-chlorobenzonitrile that replaced 2-aminobenzonitrile.

### 2. Preparation of Compound 12

Compound 12 was obtained according to the method in Example 1 with 2-cyano-4-chlorophenylhydrazine that replaced 2-cyanophenylhydrazine and with methyl 4-methoxy-3-oxobutanoate that replaced methyl 4-hydroxy-3-oxobutanoate. (ESI-MS: m/z 264.7 [M+1]⁺).

### Example 13

### Preparation of 2-(methoxymethyl)-7-methoxy-5H-benzo[d]pyrazolo[5,1-b][1,3]oxazin-5-imine (compound 13)

### 1. Preparation of 2-cyano-4-methoxyphenylhydrazine

The 2-cyano-4-methoxyphenylhydrazine was prepared with reference to the method in Example 1 in the Chinese Patent No. CN101508696A with 2-amino-5-methoxybenzonitrile that replaced 2-aminobenzonitrile.

### 2. Preparation of Compound 13

Compound 13 was obtained according to the method in Example 1 with 2-cyano-4-methoxyphenylhydrazine that replaced 2-cyanophenylhydrazine and with methyl 4-methoxy-3-oxobutanoate that replaced methyl 4-hydroxy-3-oxobutanoate.

ESI-MS: m/z 260.3 [M+1]⁺.

### Example 14

### Preparation of 2-(methoxymethyl)-7-ethoxy-5H-benzo[d]pyrazolo[5,1-b][1,3]oxazin-5-imine (compound 14)

### 1. Preparation of 2-cyano-4-ethoxyphenylhydrazine

The 2-cyano-4-ethoxyphenylhydrazine was prepared with reference to the method in the Chinese Patent No. CN101508696A with 2-amino-5-ethoxybenzonitrile that replaced 2-aminobenzonitrile.

### 2. Preparation of Compound 14

Compound 14 was obtained according to the method in Example 1 with 2-cyano-4-ethoxyphenylhydrazine that replaced 2-cyanophenylhydrazine and with methyl 4-methoxy-3-oxobutanoate that replaced methyl 4-hydroxy-3-oxobutanoate.

ESI-MS: m/z 274.3 [M+1]⁺.

### Example 15

### Preparation of 2-(ethoxymethyl)-7-fluoro-5H-benzo[d]pyrazolo[5,1-b][1,3]oxazin-5-imine (Compound 15)

Compound 15 was obtained according to the method in Example 1 with 2-cyano-4-fluorophenylhydrazine that replaced 2-cyanophenylhydrazine and with methyl 4-ethoxy-3-oxobutanoate that replaced methyl 4-hydroxy-3-oxobutanoate.

ESI-MS: m/z 262.3 [M+1]⁺.

### Example 16

### Preparation of 2-(propoxymethyl)-7-fluoro-5H-benzo[d]pyrazolo[5,1-b][1,3]oxazin-5-imine (Compound 16)

Compound 16 was obtained according to the method in Example 1 with 2-cyano-4-fluorophenylhydrazine that replaced 2-cyanophenylhydrazine and with methyl 4-propoxy-3-oxobutanoate that replaced methyl 4-hydroxy-3-oxobutanoate.

ESI-MS: m/z 276.3 [M+1]⁺.

### Example 17

### Preparation of 2-(methoxyethyl)-8-fluoro-5H-benzo[d]pyrazolo[5,1-b][1,3]oxazin-5-imine (Compound 17)

Compound 17 was obtained according to the method in Example 1 with 2-cyano-5-fluorophenylhydrazine that replaced 2-cyanophenylhydrazine and with methyl 5-methoxy-3-oxovalerate that replaced methyl 4-hydroxy-3-oxobutanoate.

ESI-MS: m/z 262.3 [M+1]⁺.

### Example 18

### Preparation of 2-(methoxypropyl)-8-fluoro-5H-benzo[d]pyrazolo[5,1-b][1,3]oxazin-5-imine (Compound 18)

### 1. Preparation of methyl 6-methoxy-3-oxohexanoate

The methyl 6-methoxy-3-oxohexanoate was prepared according to the method in Example 7 with methyl 4-methoxybutyrate that replaced methyl 2-(methylthio)acetate.

### 2. Preparation of Compound 18

Compound 18 was obtained according to the method in Example 1 with 2-cyano-5-fluorophenylhydrazine that replaced 2-cyanophenylhydrazine and with methyl 6-methoxy-3-oxohexanoate that replaced methyl 4-hydroxy-3-oxobutanoate. ESI-MS: m/z 276.3 [M+1]⁺.

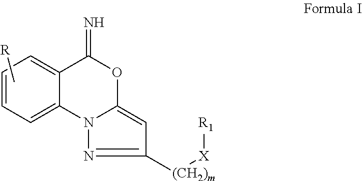

TABLE 1
Benzopyrazolooxazine derivative

| Compound | | | | |
|---|---|---|---|---|
| Number | R | m | X | R₁ |
| 1 | H | 1 | O | H |
| 2 | H | 1 | O | CH₃ |
| 3 | H | 1 | O | CH₂CH₃ |
| 4 | H | 1 | O | CH₂CH₂CH₃ |
| 5 | H | 1 | O | CHO |
| 6 | H | 1 | O | COCH₃ |
| 7 | H | 1 | O | CH₃ |
| 8 | 8-CH₃ | 1 | O | CH₃ |
| 9 | 8-CH₂CH₃ | 1 | O | CH₃ |
| 10 | 8-F | 1 | O | CH₃ |
| 11 | 7-F | 1 | O | CH₃ |
| 12 | 7-C1 | 1 | O | CH₃ |
| 13 | 7-CH₃O | 1 | O | CH₃ |
| 14 | 7-CH₃CH₂O | 1 | O | CH₃ |
| 15 | 7-F | 1 | O | CH₂CH₃ |
| 16 | 7-F | 1 | O | CH₂CH₂CH₃ |
| 17 | 8-F | 2 | O | CH₃ |
| 18 | 8-F | 3 | O | CH₃ |

### Example 19 Pharmacologic and Pharmacodynamic Bioactivity Tests

### A. Effect of Compound 2 on a PP2A Protein Expression Level in PC12 Cells

PC12 cells were cultured adherently. 1 μmol/L Aβ, 5 μmol/L Aβ, 10 μmol/L Aβ, 20 μmol/L Aβ, and 40 μmol/L Aβ were added respectively. Then, the cells were incubated for 24 h. Total protein was extracted. PP2A activity was detected by the Western Blot assay. The experimental results are shown in Table 2 and indicate that Aβ≤10 μmol/L has little effect on the expression of PP2A in PC12 cells; Aβ>10 μmol/L can significantly reduce the PP2A expression level in PC12 cells; and when the concentration of Aβ is 10 μmol/L to 40 μmol/L, Aβ inhibits the PP2A activity dose-dependently.

PC12 cells were cultured adherently. 0.1 μmol/L compound 2, 0.5 μmol/L compound 2, 1 μmol/L compound 2, 5 μmol/L compound 2, and 10 μmol/L compound 2 were added respectively. Then, the cells were incubated for 2 h. 40 μmol/L Aβ was then added. Then, the cells were incubated for 24 h. Total protein was extracted. PP2A activity was detected by the Western Blot assay. The experimental results are shown in Table 2 and indicate that compound 2 at doses of 0.1 μmol/L, 0.5 μmol/L, 1 μmol/L, and 5 μmol/L can increase the PP2A protein expression level in PC12 cells that is reduced due to Aβ, in a specific dose-effect relationship. Compound 2 at a dose of 10 μmol/L can significantly increase the PP2A protein expression level (58.59%), which has almost no difference from a PP2A expression level (58.95%) in normal cells.

TABLE 2
Effects of Aβ and compound 2 on the PP2A protein expression level in PC12 cells

| Sample | 1 | 2 | 3 | 4 | 5 | 6 | 7 | 8 | 9 | 10 | 11 |
|---|---|---|---|---|---|---|---|---|---|---|---|
| Aβ (μmol/L) | 0 | 1 | 5 | 10 | 20 | 40 | 40 | 40 | 40 | 40 | 40 |
| Counpound 2 | 0 | 0 | 0 | 0 | 0 | 0 | 0.1 | 0.5 | 1 | 5 | 10 |
| (μmol/L) | | | | | | | | | | | |
| Protein (μg/μL) | 3.15 | 2.89 | 2.67 | 2.91 | 2.78 | 2.80 | 3.06 | 2.60 | 2.84 | 2.62 | 2.73 |
| PP2A | 2069 | 1606.5 | 1628.1 | 1498.8 | 1018.9 | 914.16 | 1025.7 | 1129.7 | 1180.1 | 1284.7 | 2192.1 |
| β-actin | 3509.5 | 3563.5 | 3711.3 | 3533.8 | 3423.5 | 3354.1 | 3283.7 | 3404.8 | 3521.7 | 3607.8 | 3741.2 |
| β-actin(%) | 58.95 | 45.08 | 43.87 | 42.41 | 29.76 | 27.26 | 31.24 | 33.18 | 33.51 | 35.61 | 58.59 |

### B. Effects of Compound 1 and Compound 2 on PP2A Activity in PC12 Cells

PC12 cells were inoculated into a T25 culture flask. After the cells were attached to the wall, compound 1 and compound 2 were added, and then the cells were incubated for 2 h. 40 nmol/L okadaic acid was then added. Then, the cells were incubated for 24 h. The protein was extracted. Based on the Western Blot assay, the PP2A protein expression level in PC12 cells was detected through SDS-PAGE and transfer, immunoblotting, color development, and G: BOX chemiXR5 imaging.

The experimental results are shown in Table 3 and indicate that compared with a negative control (with only okadaic acid added), compound 1 and compound 2 at doses of 1 μmol/L, 5 μmol/L, and 10 μmol/L can significantly increase the PP2A expression level dose-dependently.

TABLE 3
Effects of compound 1 and compound 2 on the okadaic acid-
induced PP2A protein expression level in PC12 cells

| Group | Control | | 1 | | | 2 | | |
|---|---|---|---|---|---|---|---|---|
| Compound (μmol/L) | 0 | 0 | 1 | 5 | 10 | 1 | 5 | 10 |
| Okadaic Acid | 0 | 40 | 40 | 40 | 40 | 40 | 40 | 40 |
| (nmol/L) | | | | | | | | |
| PP2A | 1330.4 | 339.39 | 437.09 | 625.9 | 953.23 | 557.1 | 688.01 | 1130.4 |
| β-actin | 3616.5 | 4062.7 | 3128.5 | 3702.4 | 3768 | 3933.6 | 3838.5 | 3874.6 |
| PP2A(%) | 37 | 8 | 14 | 17 | 22 | 14 | 18 | 29 |

### C₁. Effect of Compound 2 on Phosphorylation of Tau Protein in Human Neuroblastoma Cells (SH-SY5Y Cells)

SH-SY5Y cells were cultured in vitro for 24 h in culture media containing different concentrations of compound 2. Then, the cells were treated with 40 nmol/L okadaic acid for 24 h. The p-tauSer396 level of the SH-SY5Y cells was measured by the Western Blot assay.

The experimental results are shown in Table 4 and indicate that compared with a negative control group (with only okadaic acid added), compound 2 can significantly inhibit okadaic acid-induced phosphorylation of tau protein in cells. When the dose of compound 2 is 1.0 μmol/L and 3.0 μmol/L, the p-tauSer396 level in cells decreases significantly (P<0.05). When the dose of compound 2 is 10 μmol/L, the p-tauSer396 level in cells decreases significantly (P<0.01).

TABLE 4
Effect of compound 2 on okadaic acid-induced p-tau^{Ser396}
in SH-SY5Y cells (x̅ = SEM, n = 3)

| Normal Control Group | p-tau^{Ser396}level (% ) | |
|---|---|---|
| Normal Control Group | | 0.450 ± 0.021** |
| Okadaic Acid-Induced Model Group | | 0.855 ± 0.023 |
| (Negative Control) | | |
| Compound 2 | 0.1 | 0.761 ± 0.008 |
| (μmol/L) | 0.3 | 0.663 ± 0.027 |
| | 1.0 | 0.557 ± 0.013* |
| | 3.0 | 0.578 ± 0.025* |
| | 10 | 0.522 ± 0.016** |

Compared with the negative control group, *P<0.05; **P<0.01.

### D. Effects of Compounds on a Survival Rate of Cells in Okadaic Acid- and Aβ₁₋₄₂-Induced Injury Models

### 1. Experimental Method

PC12 cells were detached and counted. A cell suspension was prepared at 8.0×10⁴ cells/mL. 100 μL of the cell suspension was added to each well of a 96-well cell culture plate. The 96-well cell culture plate was placed in an incubator at 37° C. in 5% CO₂ and cultured for 24 h. In an experimental group, the PC12 cells were pre-incubated for 2 h in a culture medium containing a test compound (the concentrations of the compound were 0.1 μmol/L, 0.5 μmol/L, 1 μmol/L, 5 μmol/L, and 10 μmol/L respectively, and the concentrations of the compound were set according to the concentrations in Table 5, Table 6, Table 7, and Table 8). Without removing the test compound, 50 nmol/L okadaic acid or 40 μmol/L Aβ₁₋₄₂ was added to act for 24 h. In addition, a negative control group (no test compound or okadaic acid added) and a model group (only okadaic acid added) were set. The 96-well cell culture plate was placed in an incubator at 37° C. in 5% CO₂ and cultured for 24 h. The cells in the 96-well cell culture plate were stained with 10 μL of CCK-8 in each well, and then cultured in the incubator for 1 h to 2 h. The 96-well cell culture plate was shaken gently for 10 min to mix uniformly and remove bubbles from the 96-well cell culture plate. The OD value of each well at 2=450 nm was read by using a microplate reader. A survival rate of cells was calculated.

Calculation formula for the cell survival rate:

$$Cell survival rate ( \% ) = \frac{OD value of the experimental group}{OD value of the negative control group} \times 100 \%$$

### 2. Experimental Results

The effect of each compound on the survival rate of PC12 cells in the okadaic acid- and Aβ₁₋₄₂-induced injury models was determined by the CCK-8 cell proliferation assay. The results are shown in Table 5 to Table 8.

It can be learned from Table 5 that each compound at 5 μmol/L significantly increases the survival rate of cells in the okadaic acid-induced injury model (P<0.01). It can be learned from Table 6 that compared with the cell survival rate of the okadaic acid model group, the survival rates of the cells treated with compound 1 at 0.5 μmol/L, 1 μmol/L, 5 μmol/L, and 10 μmol/L have significant statistical differences (P<0.01), and the survival rate of the cells treated with compound 1 at a low dose (0.1 μmol/L) has a statistical difference (P<0.05). It can be learned from Table 7 that compared with the cell survival rate of the okadaic acid model group, the survival rates of the cells treated with compound 2 at 1 μmol/L, 5 μmol/L, and 10 μmol/L have significant statistical differences (P<0.01), and the survival rates of the cells treated with compound 2 at low doses (0.1 μmol/L and 0.5 μmol/L) have no statistical difference (P>0.05).

It can be learned from Table 8 that compared with the cell survival rate of the Aβ₁₋₄₂ model group, the survival rates of the cells treated with compound 2 at 1 μmol/L, 5 μmol/L, and 10 μmol/L have statistical differences (P<0.01), and the survival rates of the cells treated with compound 2 at low doses (0.1 μmol/L and 0.5 μmol/L) have no statistical difference (P>0.05).

TABLE 5

| Effects of different compounds (5 μmol/L) on the survival | | |
|---|---|---|
| rate of cells in the okadaic acid-induced injury model | | |
| Group | Mean ± SD | Survival rate % |
| Negative control | 1.299 ± 0.023 | 100 |
| Okadaic Acid | 0.643 ± 0.014 | 49.50 |
| (50 nmol/L) | | |
| Compound 1 | 0.916 ± 0.017* | 70.52 |
| Compound 2 | 0.92 ± 0.008* | 70.82 |
| Compound 3 | 0.858 ± 0.009* | 66.05 |
| Compound 4 | 0.856 ± 0.012* | 65.90 |
| Compound 5 | 0.852 ± 0.004* | 65.59 |
| Compound 6 | 0.889 ± 0.007* | 68.44 |
| Compound 7 | 0.919 ± 0.008* | 70.75 |
| Compound 8 | 0.877 ± 0.009* | 67.51 |
| Compound 9 | 0.852 ± 0.010* | 65.59 |
| Compound 10 | 0.842 ± 0.008* | 64.82 |
| Compound 11 | 0.857 ± 0.007* | 65.97 |
| Compound 12 | 0.866 ± 0.003* | 66.67 |
| Compound 13 | 0.879 ± 0.009* | 67.67 |
| Compound 14 | 0.866 ± 0.011* | 66.67 |
| Compound 15 | 0.872 ± 0.012* | 67.13 |
| Compound 16 | 0.869 ± 0.005* | 66.90 |
| Compound 17 | 0.882 ± 0.005* | 67.90 |
| Compound 18 | 0.851 ± 0.007* | 65.55 |

Compared with okadaic acid, *P<0.01.

TABLE 6
Effects of compound 1 at different concentrations on the
survival rate of cells in the okadaic acid-induced injury model

| Group | | Mean ± SD | Survival rate % |
|---|---|---|---|
| Negative control | | 2.297 ± 0.016 | 100 |
| Okadaic Acid (50 nmol/L) | | 1.225 ± 0.018 | 53.33 |
| Compound 1 | 10 | 1.76 ± 0.032** | 76.62 |
| (μmol/L) | 5 | 1.647 ± 0.028** | 71.70 |
| | 1 | 1.449 ± 0.013** | 63.08 |
| | 0.5 | 1.382 ± 0.013** | 60.17 |
| | 0.1 | 1.337 ± 0.008* | 58.21 |

Compared with okadaic acid, *P<0.05; **P<0.01.

TABLE 7
Effects of compound 2 at different concentrations on the
survival rate of cells in the okadaic acid-induced injury model

| Group | | Mean ± SD | Survival rate % |
|---|---|---|---|
| Negative control | | 1.326 ± 0.021 | 100 |
| Okadaic Acid (50 nmol/L) | | 0.669 ± 0.025 | 50.45 |
| Compound 2 | 10 | 1.04 ± 0.017* | 78.43 |
| (μmol/L) | 5 | 0.967 ± 0.018* | 72.93 |
| | 1 | 0.787 ± 0.028* | 59.35 |
| | 0.5 | 0.676 ± 0.016 | 50.98 |
| | 0.1 | 0.652 ± 0.018 | 49.17 |

Compared with okadaic acid, *P<0.01.

TABLE 8

| Effects of compound 2 at different concentrations on the | | | |
|---|---|---|---|
| survival rate of cells in the AB1-42-induced injury model | | | |
| Group | | Mean ± SD | Survival rate % |
| Negative control | | 1.378 ± 0.017 | 100 |
| Aβ₁₋₄₂ (40 μmol/L) | | 0.709 ± 0.023 | 51.45 |
| Compound 2 | 10 | 1.09 ± 0.026* | 79.10 |
| (μmol/L) | 5 | 0.941 ± 0.018* | 68.29 |
| | 1 | 0.768 ± 0.015* | 55.73 |
| | 0.5 | 0.698 ± 0.024 | 50.65 |
| | 0.1 | 0.699 ± 0.027 | 50.73 |

Compared with Aβ₁₋₄₂, *P < 0.01.

What is claimed is:

1. A benzopyrazolooxazine derivative of a structure shown in formula I, or a pharmaceutically acceptable salt thereof: 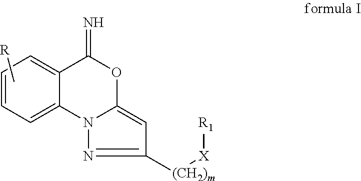
wherein
R is selected from hydrogen, halogen, alkyl, or alkoxy;
m is an integer from 1 to 3;
X is selected from oxygen or sulfur; and
R₁ is selected from hydrogen, alkyl, or acyl.

2. The benzopyrazolooxazine derivative according to claim 1, wherein R is selected from hydrogen, halogen, C₁-C₂ alkyl, or C₁-C₂ alkoxy;
m is an integer from 1 to 3;
X is selected from oxygen or sulfur; and
R₁ is selected from hydrogen, C₁-C₃ alkyl, formyl, or acetyl.

3. The benzopyrazolooxazine derivative according to claim 1, selected from the following compounds:
(5-imino-5H-benzo[d]pyrazolo[5,1-b][1,3]oxazin-2-yl) methanol;
2-(methoxymethyl)-5H-benzo[d]pyrazolo[5,1-b][1,3]oxazin-5-imine;
2-(ethoxymethyl)-5H-benzo[d]pyrazolo[5,1-b][1,3]oxazin-5-imine;
2-(propoxymethyl)-5H-benzo[d]pyrazolo[5,1-b][1,3]oxazin-5-imine;
(5-imino-5H-benzo[d]pyrazolo[5,1-b][1,3]oxazin-2-yl)methyl formate;
(5-imino-5H-benzo[d]pyrazolo[5,1-b][1,3]oxazin-2-yl)methyl acetate;
2-((methylthio)methyl)-5H-benzo[d]pyrazolo[5,1-b][1,3]oxazin-5-imine;
2-(methoxymethyl)-8-methyl-5H-benzo[d]pyrazolo[5,1-b][1,3]oxazin-5-imine;
2-(methoxymethyl)-8-ethyl-5H-benzo[d]pyrazolo[5,1-b][1,3]oxazin-5-imine;
2-(methoxymethyl)-8-fluoro-5H-benzo[d]pyrazolo[5,1-b][1,3]oxazin-5-imine;
2-(methoxymethyl)-7-fluoro-5H-benzo[d]pyrazolo[5,1-b][1,3]oxazin-5-imine;
2-(methoxymethyl)-7-chloro-5H-benzo[d]pyrazolo[5,1-b][1,3]oxazin-5-imine;
2-(methoxymethyl)-7-methoxy-5H-benzo[d]pyrazolo[5,1-b][1,3]oxazin-5-imine;
2-(methoxymethyl)-7-ethoxy-5H-benzo[d]pyrazolo[5,1-b][1,3]oxazin-5-imine;
2-(ethoxymethyl)-7-fluoro-5H-benzo[d]pyrazolo[5,1-b][1,3]oxazin-5-imine;
2-(propoxymethyl)-7-fluoro-5H-benzo[d]pyrazolo[5,1-b][1,3]oxazin-5-imine;
2-(methoxyethyl)-8-fluoro-5H-benzo[d]pyrazolo[5,1-b][1,3]oxazin-5-imine; and
2-(methoxypropyl)-8-fluoro-5H-benzo[d]pyrazolo[5,1-b][1,3]oxazin-5-imine.

4. A medicament for prevention and treatment of a disease caused by lack of PP2A activity or reduction of PP2A functions, comprising the benzopyrazolooxazine derivative according to claim 1.

5. The medicament according to claim 4, wherein the disease caused by lack of PP2A activity or reduction of PP2A functions is a tumor.

6. The medicament according to claim 4, wherein the disease caused by lack of PP2A activity or reduction of PP2A functions is a degenerative brain disease.

7. The medicament according to claim 6, wherein the degenerative brain disease is Alzheimer's disease.

8. A protein phosphatase agonist comprising the benzopyrazolooxazine derivative according to claim 1.

9. A protein phosphatase 2A agonist comprising the benzopyrazolooxazine derivative according to claim 1.
